(19) United States
(12) Patent Application Publication
(10) Pub. No.: US 2025/0090767 A1
(43) Pub. Date: Mar. 20, 2025

(54) Safety Device for a Syringe

(71) Applicant: Plasto Design Ltd, Holon (IL)

(72) Inventor: Boaz Zer, Levahim (IL)

(73) Assignee: Plasto Design Ltd, Holon (IL)

(21) Appl. No.: 18/882,235

(22) Filed: Sep. 11, 2024

Related U.S. Application Data
(60) Provisional application No. 63/583,363, filed on Sep. 18, 2023.

(51) Int. Cl. A61M 5/32

(52) U.S. Cl. CPC A61M 5/326; A61M 2005/325

(57) ABSTRACT

The present invention relates to a safety device (100) for a syringe assembly (302), for efficiently preventing accidental usage of the syringe assembly (302) for administration of a drug to a patient. The safety device (100) includes a tubular body (102) having a distal end (102-1) and a proximal end (102-2), and a spring-loaded cap (104). The cap (104) may be inserted into a hollow cavity of the body (102) at its proximal end (102-2). In a retracted position, movement of the cap (104) is restricted with respect to the proximal end (102-2). The safety device (100) also includes an actuation member (106) operably coupled with the cap (104) to switch from the retracted position to an extended position, in which the needle (304) is positioned inside the hollow cavity of the body (102) and is fully covered by the body (102).

### TECHNICAL FIELD

The present invention relates to syringes, and more particularly to a safety device for a syringe capable of safe and efficient administration of drugs.

### BACKGROUND OF INVENTION

Background description includes information that may be useful in understanding the present invention. It is not an admission that any of the information provided herein is prior art or relevant to the present invention, or that any publication specifically or implicitly referenced is prior art.

Prevention of needle sticks is of paramount concern in the healthcare industry because of serious and deadly risk factors associated with certain transmittable or communicable diseases. Typical injection and/or blood collection devices utilize a needle which can be, among other things, inserted into a patient so as to inject a substance or draw a fluid such as blood through the needle into an associated separate collection reservoir.

Accidental needle sticks from currently or previously used needles can occur during the process of injection and during the fluid withdrawing process as well as during subsequent handling and disposal. Until such used medical devices are destroyed or discarded into a proper disposal collection device such as a sharps container, they remain a risk to those handling them as well as those that may come into contact with them. Most conventional devices for preventing accidental needle sticks often expose the patient to the risk of inadvertent needle sticks.

There is therefore a need to develop techniques for enabling safe handling of syringe needles for efficient administration of drug to a patient, while addressing the above-mentioned shortcoming of conventional syringes.

### OBJECTS OF THE PRESENT DISCLOSURE

It is an object of the present disclosure to provide a safety device for a syringe capable of efficient administration of drugs to a patient.

It is another object of the present disclosure to provide a safety device for allowing safe handling of syringe while preventing inadvertent use of syringe needles.

It is another object of the present disclosure to provide a safety device for a syringe capable of being easily functioning with existing syringes.

### SUMMARY OF THE INVENTION

An aspect of the present invention relates to a safety device for a syringe, capable of effectively protecting a syringe needle from inadvertently contaminating a drug to be administered to a patient, while preventing accidental application of the syringe needle for safe administration of the drug. The safety device includes a tubular body having a distal end and a proximal end. The body may have a hollow cavity capable of receiving and accommodating a syringe assembly. The syringe assembly may be inserted into the hollow cavity of the body through the distal end. The safety device also includes a spring-loaded cap. The cap may be inserted into the hollow cavity of the body at the proximal end. In a retracted position, movement of the cap is restricted with respect to the proximal end of the body. The safety device also includes an actuation member operably coupled with the cap to switch from the retracted position to an extended position, in which a needle of the syringe assembly is positioned inside the hollow cavity of the body and is fully covered by the body.

According to an embodiment of the present disclosure, the tubular body may include a plurality of internal lugs at the distal end for fixedly holding the syringe assembly.

According to an embodiment of the present disclosure, the needle of the syringe assembly may be attached to a luer slip of the syringe assembly after the syringe assembly is inserted into the hollow cavity of the tubular body

According to an embodiment of the present disclosure, the spring-loaded cap may include a first slot and a second slot located at axial ends thereof, and a plurality of teeth adapted to scratch an outer surface of the luer slip and prevent the luer slip from moving out of the spring-loaded cap.

According to an embodiment of the present disclosure, the first slot may have a larger diameter than the second slot.

According to an embodiment of the present disclosure, the spring-loaded cap may be biased with respect to the proximal end of the tubular body by a spring, to enable the spring-loaded cap to remain in the retracted position.

According to an embodiment of the present disclosure, the spring may be supported by a first flange positioned at the first end of the spring-loaded cap and a second flange located at the proximal end of the tubular body, such that in the retracted position, the spring-loaded cap is restricted to move with respect to the proximal end of the tubular body.

According to an embodiment of the present disclosure, the actuation member may include an actuation end positioned in contact with the first flange of the spring-loaded cap, to restrict the spring-loaded cap present in the retracted position to move along an axial direction of the tubular body.

According to an embodiment of the present disclosure, the actuation member may also include and a free end, which when pushed, enables the actuation end to move in a direction such that the actuation end gets disengaged from the first flange of the spring-loaded cap, to move the spring-loaded cap to the extended position.

According to an embodiment of the present disclosure, the actuation member may be in the form of a link having a curved profile between the actuation end and the free end.

According to an embodiment of the present disclosure, when the spring-loaded cap is present in the extended position, the syringe assembly may be positioned at a finger flange area located at the proximal end of the tubular body, in which the first flange is engaged with a latch of the tubular body, to prevent the syringe assembly from moving towards the distal end of the tubular body.

Various objects, features, aspects and advantages of the inventive subject matter will become more apparent from the following detailed description of preferred embodiments, along with the accompanying drawing figures in which like numerals represent like components.

### BRIEF DESCRIPTION OF ACCOMPANYING DRAWINGS

The accompanying drawings are included to provide a further understanding of the present disclosure, and are incorporated in and constitute a part of this specification. The drawings illustrate exemplary embodiments of the present disclosure and, together with the description, serve to explain the principles of the present disclosure.

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a perspective view of a safety device for a syringe, in accordance with an embodiment of the present invention;

FIG. 2 illustrates a cross-sectional view of the safety device, in accordance with an embodiment of the present invention;

FIGS. 3A to 3C illustrate cross-sectional views of the safety device provided with a syringe assembly, in accordance with an embodiment of the present invention; and

FIGS. 4 and 5 illustrate cross-sectional view of the safety device with a cap thereof present in an extended position, in accordance with an embodiment of the present invention.

### DETAILED DESCRIPTION OF THE INVENTION

The following is a detailed description of embodiments of the disclosure depicted in the accompanying drawings. The embodiments are in such details as to clearly communicate the disclosure. However, the amount of detail offered is not intended to limit the anticipated variations of embodiments; on the contrary, the intention is to cover all modifications, equivalents, and alternatives falling within the scope of the present disclosures as defined by the appended claims.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

If the specification positions a component or feature “may”, “can”, “could”, or “might” be included or have a characteristic, that particular component or feature is not required to be included or have the characteristic.

Exemplary embodiments will now be described more fully hereinafter with reference to the accompanying drawings, in which exemplary embodiments are shown. This disclosure may however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

In some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

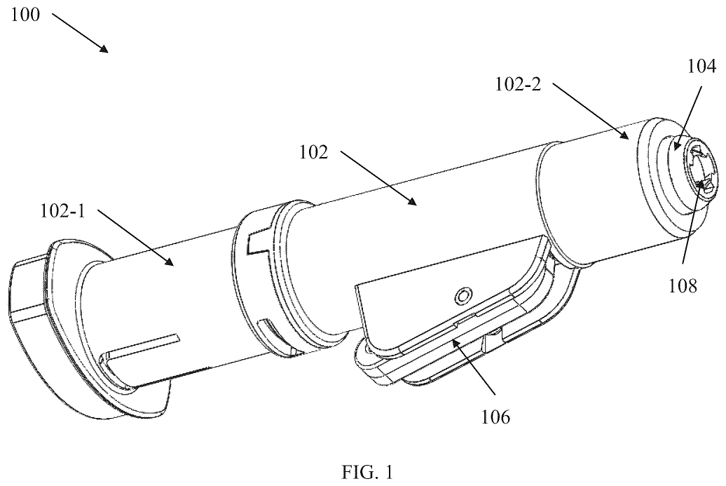

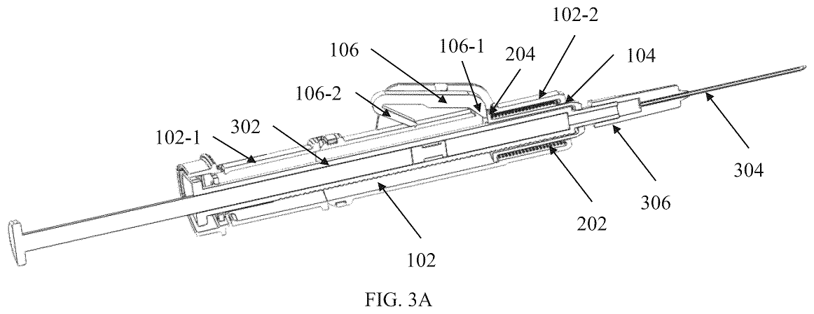

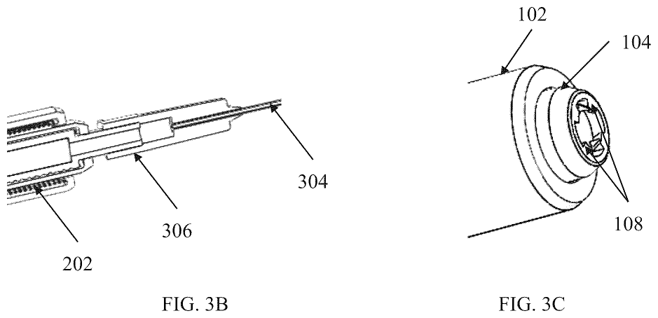

The present invention relates to a safety device for a syringe, for efficiently preventing any inadvertent usage of the syringe. The safety device ensures that the needle is single-use, and is not accidentally administered to a patient. FIG. 1 illustrates an exemplary representation of a safety device (100) for a syringe. The safety device (100) includes a cylindrical or tubular body (also referred to as “body” herein) (102) having a distal end (102-1) and a proximal end (102-2). The body (102) may have a hollow cavity capable of receiving and accommodating a syringe assembly. An internal surface of the body (102) may include a plurality of lugs at the distal end (102-1) for fixedly holding the syringe assembly, which may be passed in the hollow cavity of the body (102) through the distal end (102-1). After the syringe assembly is inserted into the hollow cavity of the body (102), a needle may be attached to a luer slip of the syringe assembly, as shown in FIG. 3A.

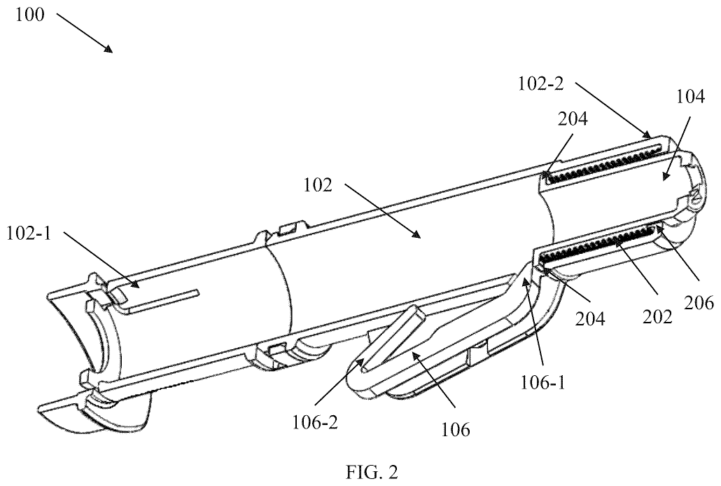

Referring now to FIG. 2, the safety device (100) also includes a cap (104) having a hollow cavity. The cap (104) may include a first slot and a second slot provided at axial ends thereof. The first slot of the cap (104) may have a bigger diameter than the second slot. The cap (104) may include a plurality of teeth (108). The cap (104) may be inserted into the hollow cavity of the body (102) at the proximal end (102-2) of the body (102). The cap (104) may be spring-loaded with respect to the proximal end (102-2) of the body (102) with the help of a coil spring (202). The coil spring (202) may be supported by a first flange (204) provided at the first end of the cap (104) and a second flange (206) provided at the proximal end (102-2) of the body (102), such that in a retracted position, movement of the cap (104) is restricted with respect to the proximal end (102-2) of the body (102).

The safety device (100) also includes an actuation member (106) having an actuation end (106-1) in contact with the first flange (204) of the cap (104). In the retracted position of the cap (104), the actuation end (106-1) prevents movement of the cap (104) along axial direction of the body (102). The actuation member (106) also includes a free end (106-2), which when pushed by a user allows the actuation end (106-1) to move in a direction such that the actuation end (106-1) gets disconnected or disengaged from the first flange (204) of the cap (104). This allows the cap (104) to move in towards the distal end (102-1) of the cap (104) under the action of the coil spring (202). The actuation member (106) may be in the form of a link having a curved profile between the actuation end (106-1) and the free end (106-2).

FIG. 3A illustrate a syringe assembly (302) inserted into the body (102) of the safety device (100). Firstly, the syringe assembly (302) is inserted into the hollow cavity of the body (102) through the distal end (102-1). The syringe assembly (302) may include a syringe and a plunger. The syringe assembly (302) is inserted and pushed inside the body (102) until the syringe is locked in position with the cap (104). Thereafter, a needle (304) of the syringe assembly (302) may be attached to the luer slip (306) of the syringe assembly (302), at the proximal end (102-2) of the cap (104). At this stage, a drug may be injected or stored inside the syringe by pulling the plunger.

Referring now to FIGS. 3B and 3C, where magnified view of the luer slip (304) attached to the cap (104) is shown. The luer slip (306) of the syringe assembly (302) may be inserted into the cap (104) by some force. The cap (104) includes a plurality of teeth (108) adapted to scratch outer surface of the luer slip (306), and hold/prevent the luer slip (306) from moving out of the cap (104). This ensures that the luer slip (306) is fixedly held by the cap (104). The needle (304) is attached to the luer slip (306) after the luer slip (306) is fixedly accommodated in the cap (104).

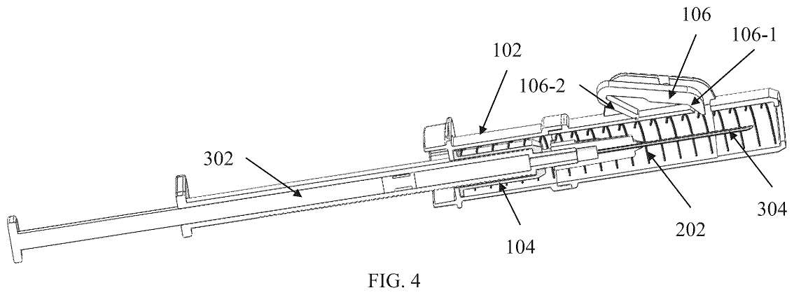

FIG. 4 illustrates an extended position of the cap (104) in which the needle (304) is positioned inside the hollow cavity of the body (102) and any accidental usage of the needle (304) is prevented. After the needle (304) is used to administer a drug to a patient, the free end (106-2) of the actuation member (106) may be pressed/pushed by the user, such as a medical personnel. This releases the actuation end (106-1) of the actuation member (106) from the first flange (204) of the cap (104), and moves the cap (104) towards the distal end (102-1) of the cap (104) under the action of the coil spring (202). In this extended position of the cap (104), the needle (304) is positioned inside the hollow cavity of the body (102) and is fully covered by the body (102). This protects the needle (304) from any accidental usage, and prevents the needle (304) to contaminate any drug to be administered to the patient.

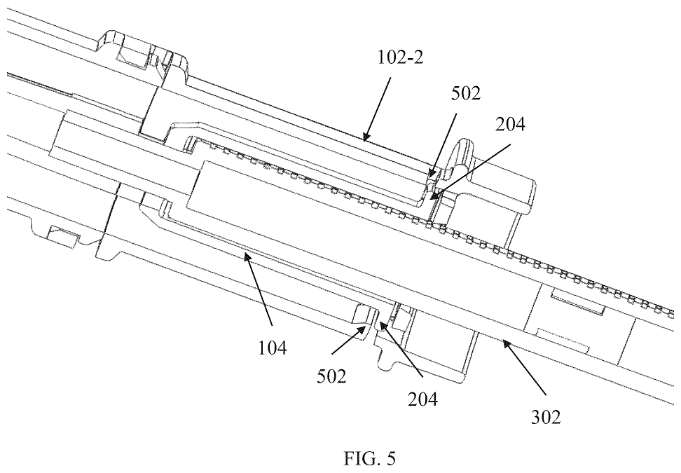

FIG. 5 illustrates a cross-sectional view of the proximal end (102-2) of the body (102) with the cap (104) being in its extended position. After activating the safety feature, i.e., in the extended position of the cap (104), the syringe assembly (302) is pushed back by the spring (202), and the syringe assembly (302) arrives at a finger flange area located at the proximal end (102-2) of the body (102). When the syringe assembly (302) arrives at a finger flange area, movement of the syringe assembly (302) is locked in a latch (502) of the body (102) such that the first flange (204) provided at the first end of the cap (104) is engaged with the latch (502). This prevents the syringe assembly (302) from moving towards the distal end (102-1) of the body (102), thereby locking movement of the needle (304) inside the hollow cavity of the body (102) and preventing any accidental usage thereof.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

1. A safety device (100) for a syringe assembly (302), comprising:
a tubular body (102) having a distal end (102-1) and a proximal end (102-2), wherein the tubular body (102) includes a hollow cavity for receiving the syringe assembly (302) through the distal end (102-1) thereof;
a spring-loaded cap (104) inserted into the hollow cavity at the proximal end (102-2) of the tubular body (102); and
an actuation member (106) configured to move the spring-loaded cap (104) between a retracted position and an extended position, wherein in the retracted position, the spring-loaded cap (104) is restricted to move with respect to the proximal end (102-2) of the tubular body (102), and in the extended position of the spring-loaded cap (104), a needle (304) of the syringe assembly (302) is positioned inside the hollow cavity of the tubular body (102).

2. The safety device (100) as claimed in claim 1, wherein the tubular body (102) comprises a plurality of internal lugs at the distal end (102-1) for fixedly holding the syringe assembly (302).

3. The safety device (100) as claimed in claim 1, wherein the needle (304) of the syringe assembly (302) is attached to a luer slip of the syringe assembly (302) after the syringe assembly (302) is inserted into the hollow cavity of the tubular body (102).

4. The safety device (100) as claimed in claim 3, wherein the spring-loaded cap (104) comprises a first slot and a second slot located at axial ends thereof, and a plurality of teeth (108) adapted to scratch an outer surface of the luer slip (306) and prevent the luer slip (306) from moving out of the spring-loaded cap (104).

5. The safety device (100) as claimed in claim 4, wherein the first slot has a larger diameter than the second slot.

6. The safety device (100) as claimed in claim 1, wherein the spring-loaded cap (104) is biased with respect to the proximal end (102-2) of the tubular body (102) by a spring (202), to enable the spring-loaded cap (104) to remain in the retracted position.

7. The safety device (100) as claimed in claim 6, wherein the spring (202) is supported by a first flange (204) positioned at the first end of the spring-loaded cap (104) and a second flange (206) located at the proximal end (102-2) of the tubular body (102), such that in the retracted position, the spring-loaded cap (104) is restricted to move with respect to the proximal end (102-2) of the tubular body (102).

8. The safety device (100) as claimed in claim 7, wherein the actuation member (106) comprises:
an actuation end (106-1) positioned in contact with the first flange (204) of the spring-loaded cap (104), to restrict the spring-loaded cap (104) present in the retracted position to move along an axial direction of the tubular body (102); and
a free end (106-2), which when pushed, enables the actuation end (106-1) to move in a direction such that the actuation end (106-1) gets disengaged from the first flange (204) of the spring-loaded cap (104), to move the spring-loaded cap (104) to the extended position.

9. The safety device (100) as claimed in claim 8, wherein the actuation member (106) is in the form of a link having a curved profile between the actuation end (106-1) and the free end (106-2).

10. The safety device (100) as claimed in claim 1, wherein when the spring-loaded cap (104) is present in the extended position, the syringe assembly (302) is positioned at a finger flange area located at the proximal end (102-2) of the tubular body (102), in which the first flange (204) is engaged with a latch (502) of the tubular body (102), to prevent the syringe assembly (302) from moving towards the distal end (102-1) of the tubular body (102).
